(19) United States
(12) Patent Application Publication
(10) Pub. No.: US 2018/0100091 A1
(43) Pub. Date: Apr. 12, 2018

(54) HOT MELT ADHESIVES AND DISPOSABLE PRODUCTS USING THE SAME

(71) Applicant: Sekisui Fuller Company, Ltd., Tokyo (JP)

(72) Inventors: Tetsuyasu SUGIMOTO, Shizuoka (JP); Yu SOMEYA, Shizuoka (JP); Rikio GENMA, Shizuoka (JP); Munetaka YOSHII, Shizuoka (JP); Mamoru KOIKE, Shizuoka (JP); Kazuya SUZUKI, Shizuoka (JP)

(73) Assignee: Sekisui Fuller Company, Ltd., Tokyo (JP)

(21) Appl. No.: 15/567,130

(22) PCT Filed: Apr. 22, 2016
(86) PCT No.: PCT/JP2016/002157

§ 371 Date: Oct. 17, 2017

(30) Foreign Application Priority Data
Apr. 23, 2015 (JP) 2015-088808

(51) Int. Cl. C09J 153/02; C09J 11/06; A61L 15/58

(52) U.S. Cl. CPC C09J 153/02; A61L 15/585; C09J 11/06

(57) ABSTRACT

The present invention provides hot melt adhesives. A hot melt adhesive according to the present invention contains a styrenic block copolymer (A) containing a styrene-butadiene-styrene block copolymer (A1); and at least one styrenic block copolymer (A4) selected from the group consisting of a styrene-butadiene-styrene block copolymer (A2) and a styrene-isoprene-styrene block copolymer (A3), wherein the styrenic block copolymer (A) contains a predetermined amount of the styrene-butadiene-styrene block copolymer (A1) relative to the total amount of the styrene-butadiene-styrene block copolymer (A1) and the styrenic block copolymer (A4); a tackifier (B); and a plasticizer (C), wherein the hot melt adhesive has a melt viscosity of 6000 mPa s or lower.

### TECHNICAL FIELD

The present invention relates to low-temperature hot melt adhesives that can be applied at relatively low temperatures, with good tack at low temperatures and with adhesive strength and holding power at room temperature not being impaired. More particularly, the present invention relates to hot melt adhesives that can be used suitably for the production of disposable products such as disposable diapers.

### BACKGROUND ART

Some disposable products, such as disposable diapers and sanitary napkins, have been conventionally made of components including a polyolefin resin film, a non-woven fabric, a tissue paper, and a natural rubber. These components are fixed with a hot melt adhesive to produce the disposable products.

Typical hot melt adhesives used for disposable products of this kind include: rubber-based adhesives containing as a major ingredient a thermoplastic block copolymer of a vinyl aromatic hydrocarbon and a conjugated diene compound, and polyolefin adhesives containing as a major ingredient an ethylene-propylene copolymer. Rubber-based adhesives are more widely used than polyolefin adhesives because of their better usability and cohesive strength.

Hot melt adhesives are melted with heat in a hot melt tank before being applied to subjects. The molten adhesive is applied either in contact or non-contact fashion. Slot coating and roll coating are examples of the contact application techniques. Swirl pattern application, omega-shaped pattern application, and curtain spraying are examples of the non-contact application techniques. The non-contact application techniques are more often used because they allow an appropriate adhesive strength with a relatively small amount of hot melt adhesive and do not harm the texture of disposable products.

Rubber-based hot melt adhesives used for the production of disposable products such as disposable diapers and sanitary napkins typically contain 15-35% by mass of polymer components to provide appropriate adhesive strength and holding power. Furthermore, a melt viscosity suitable for hot melt applications is provided by blending a tackifier and a plasticizer. Since such hot melt adhesives typically have a melt viscosity at 160° C. of 3,000 mPa s or higher, they are melted at 150-170° C. and applied to various substrates when in use.

Hot melt adhesives, when applied to a substrate at high temperatures of 150-170° C., can thermally damage the substrate by their heat and spoil the appearance and/or properties of disposable products such as disposable diapers and sanitary napkins. In addition, it is desirable that hot melt adhesives are applied at a lower temperature, preferably at 120° C. or lower in light of the safety of workers who use hot melt adhesives.

It is contemplated that, in order to allow application of a hot melt adhesive at a relatively low temperature, the amount of the styrenic block copolymer blended with the adhesive will be reduced to lower the melt viscosity of that adhesive. This approach, however, also reduces the holding power of the hot melt adhesive, which could possibly result in significant deterioration of the properties as a hot melt adhesive.

As described above, hot melt adhesives are usually melted in a hot melt tank before being applied to subjects. If the adhesives have poor thermal stability, they could possibly be degraded in the tank, leading to the reduction in their melt viscosity. In such a case, the affected hot melt adhesives cannot provide their intended properties. Accordingly, hot melt adhesives having good thermal stability are desired in the production of disposable products.

PLT 1 discloses hot melt adhesives having improved holding power with a smaller amount of styrenic block copolymer.

PLT 2 discloses hot melt adhesives which can be applied at a relatively low temperature of about 110-130° C. and are suitable for bonding an elastic attachment in disposable products.

### CITATION LIST

### Patent Literature

PTL 1: JP-A 2008-239931

PTL 2: JP-T-2014-508832

### SUMMARY OF INVENTION

### Technical Problem

The hot melt adhesives disclosed in PLT 1, however, have a melt viscosity at 160° C. of 3500 mPa s or higher and are thus not suitable for non-contact applications performed at a temperature of 120° C. or lower. Accordingly, there is a need for hot melt adhesives having a low melt viscosity which can be applied in a non-contact fashion at a temperature of 120° C. or lower without losing their appropriate adhesive strength and holding power as a hot melt adhesive.

The hot melt adhesives disclosed in PLT 2 contain only a styrene-isoprene-styrene block copolymer as the polymer component. In general, styrene-isoprene-styrene block copolymers are likely to suffer from molecular cleavage by heat and, therefore, improvement of their thermal stability is desired.

Furthermore, when the hot melt adhesive is applied to a substrate at a relatively low temperature, a hot melt adhesive could possibly get cold, resulting in decrease of its tack, before the substrate is bonded to another substrate. The decrease in tack due to the cooling of the hot melts occurs more rapidly in winter season. Hot melt adhesives with a lower tack would provide inappropriate bonding between the substrates, causing a problem in the production of disposable products such as disposable diapers. Accordingly, there is a need for hot melt adhesives that can keep their tack even when applied at a relatively low temperature.

In consideration of the aforementioned problems, an object of the present invention is to provide hot melt adhesives that can be applied at a relatively low temperature without losing their tack at low temperatures and with keeping good thermal stability along with adhesive strength and holding power at room temperature.

### Solution to Problem

An aspect of the present invention is a hot melt adhesive containing: 100 parts by mass of a styrenic block copolymer (A), the styrenic block copolymer (A) containing a styrene-butadiene-styrene block copolymer (A1) having styrene content of 40-50% by mass and a viscosity in a 25% toluene solution of 100-300 mPa s, and at least one styrenic block copolymer (A4) selected from the group consisting of a styrene-butadiene-styrene block copolymer (A2) having a styrene content of 15-40% by mass and a viscosity in a 25% toluene solution of 300-3500 mPa s, and a styrene-isoprene-styrene block copolymer (A3), wherein (A1) is 30-70% by mass of the total amount of the styrene block copolymers (A1) plus (A4);

100-400 parts by mass of a tackifier (B); and

a plasticizer (C); wherein

the hot melt adhesive has a melt viscosity of 6000 mPa s or lower at 120° C.

In the aforementioned hot melt adhesive, the tackifier (B) may contain 10-100 parts by mass of a fully hydrogenated tackifier (B1) having a ring-and-ball softening point of 100° C. or lower, relative to 100 parts by mass of the styrenic block copolymer (A).

In the aforementioned hot melt adhesive, the tackifier (B) may contain an end-block tackifier (B2) having a ring-and-ball softening point of 95° C. or lower.

Another aspect of the present invention is disposable products made by using the hot melt adhesive described above.

### Advantageous Effects of Invention

The hot melt adhesives according to the present invention have excellent adhesive strength and holding power at room temperature as well as excellent tack at low temperatures. Furthermore, the hot melt adhesives according to the present invention have excellent thermal stability as well as excellent usability at relatively low temperatures.

### DESCRIPTION OF EMBODIMENTS

The aforementioned hot melt adhesives are described in detail below. The hot melt adhesive according to the present invention contains a styrenic block copolymer (A), 100-400 parts by mass of tackifier (B), and a plasticizer (C), and has a melt viscosity of 6,000 mPa s or lower at 120° C.

### Styrenic Block Copolymer (A)

The hot melt adhesive according to the present invention contains the styrenic block copolymer (A).

The styrenic block copolymer (A) contains:

a styrene-butadiene-styrene block copolymer (A1) having a styrene content of 40-50% by mass and a viscosity in a 25% toluene solution of 100-300 mPa s; and

at least one styrenic block copolymer (A4) selected from the group consisting of a styrene-butadiene-styrene block copolymer (A2) having a styrene content of 15-40% by mass and a viscosity in a 25% toluene solution of 300-3500 mPa s, and a styrene-isoprene-styrene block copolymer (A3).

The styrene-butadiene-styrene block copolymer (A1) may be hydrogenated or not. It is, however, preferable that the styrene-butadiene-styrene block copolymer (A1) is not hydrogenated because the usability at low temperatures of the hot melt adhesive in which the styrene-butadiene-styrene block copolymer (A1) is not hydrogenated is excellent.

The styrene content in the styrene-butadiene-styrene block copolymer (A1) is 40-50% by mass, preferably 42-48% by mass, and more preferably 44-46% by mass. When the styrene content in the styrene-butadiene-styrene block copolymer (A1) is 40% by mass or more, the holding power of the hot melt adhesive at room temperature is improved. When the styrene content in the styrene-butadiene-styrene block copolymer (A1) is 50% by mass or less, the tack of the hot melt adhesive at low temperatures is improved. The styrene content in the styrene-butadiene-styrene block copolymer used in the present invention refers to the total percentage of styrene blocks in the styrene-butadiene-styrene block copolymer.

The viscosity of the styrene-butadiene-styrene block copolymer (A1) in a 25% toluene solution is 100-300 mPa s, preferably 120-230 mPa s, and more preferably 140-210 mPa s. It is particularly preferable that the viscosity of the styrene-butadiene-styrene block copolymer (A1) in a 25% toluene solution is 160-190 mPa s. When the viscosity of the styrene-butadiene-styrene block copolymer (A1) in a 25% toluene solution is 100 mPa s or higher, the holding power of the hot melt adhesive at room temperature is improved. When the viscosity of the styrene-butadiene-styrene block copolymer (A1) in a 25% toluene solution is 300 mPa s or lower, the usability of the hot melt adhesive at low temperatures is improved.

In the present invention, the viscosity of the styrene-butadiene-styrene block copolymer in a 25% toluene solution refers to a viscosity of a solution of 25% by mass of styrene-butadiene-styrene block copolymer in toluene at 23° C. The viscosity in a 25% toluene solution can be determined using one of various viscometers such as a Brookfield viscometer, BM-type. The spindle used for the measurement can appropriately be selected depending on the viscosity of the solution of 25% by mass of styrene-butadiene-styrene block copolymer in toluene. For example, a spindle #2 is used.

The content of the styrene-butadiene-styrene block copolymer (A1) in the hot melt adhesive is preferably 30-70% by mass, and more preferably 40-60% by mass, relative to the total amount of the styrene-butadiene-styrene block copolymer (A1) and the styrenic block copolymer (A4). When the content of the styrene-butadiene-styrene block copolymer (A1) is 30% by mass or more, the cohesive strength of the hot melt adhesive is improved, leading to improved holding power at room temperature, and its usability at low temperatures is also improved. When the content of the styrene-butadiene-styrene block copolymer (A1) is 70% by mass or less, the hot melt adhesive becomes softer, improving its tack at low temperatures.

The styrene-butadiene-styrene block copolymer (A1) is commercially available. Examples of the commercially available styrene-butadiene-styrene block copolymer (A1) include “Asaprene T-439” available from Asahi Kasei Chemicals Corporation and “Globalprene 3545” available from LCY Chemical Corporation.

The styrenic block copolymer (A4) is selected from the group consisting of the styrene-butadiene-styrene block copolymer (A2) having a styrene content of 15-40% by mass and a viscosity in a 25% toluene solution of 300-3500 mPa s, and the styrene-isoprene-styrene block copolymer (A3).

The styrenic block copolymer (A4) preferably contains the styrene-butadiene-styrene block copolymer (A2) because the thermal stability of the hot melt adhesive is improved.

The styrene-butadiene-styrene block copolymer (A2) may be hydrogenated or not. It is, however, preferable that the styrene-butadiene-styrene block copolymer (A2) is not hydrogenated because the usability at low temperatures of the hot melt adhesive in which the styrene-butadiene-styrene block copolymer (A2) is not hydrogenated is excellent.

The styrene content in the styrene-butadiene-styrene block copolymer (A2) is 15-40% by mass, preferably 20-38% by mass, and more preferably 25-37% by mass. It is particularly preferable that the styrene content in the styrene-butadiene-styrene block copolymer (A2) is 29-36% by mass. When the styrene content in the styrene-butadiene-styrene block copolymer (A2) is 15% by mass or more, the holding power of the hot melt adhesive at room temperature is improved. When the styrene content in the styrene-butadiene-styrene block copolymer (A2) is 40% by mass or less, the tack of the hot melt adhesive at low temperatures is improved.

The viscosity of the styrene-butadiene-styrene block copolymer (A2) in a 25% toluene solution is 300-3500 mPa s, preferably 350-2500 mPa s, and more preferably 400-2000 mPa s. When the viscosity of the styrene-butadiene-styrene block copolymer (A2) in a 25% toluene solution is 300 mPa s or higher, the holding power of the hot melt adhesive at room temperature is improved. When the viscosity of the styrene-butadiene-styrene block copolymer (A2) in a 25% toluene solution is 3500 mPa s or lower, the usability of the hot melt adhesive at low temperatures is improved.

The styrene-butadiene-styrene block copolymer (A2) is commercially available. Examples of the commercially available styrene-butadiene-styrene block copolymer (A2) include “Asaprene T-438” and “Asaprene T-432,” both available from Asahi Kasei Chemicals Corporation.

The content of the styrene-butadiene-styrene block copolymer (A2) in the hot melt adhesive is preferably 30-70% by mass, and more preferably 40-60% by mass, relative to the total amount of the styrene-butadiene-styrene block copolymer (A1) and the styrenic block copolymer (A4). When the content of the styrene-butadiene-styrene block copolymer (A2) is 30% by mass or more, the hot melt adhesive becomes softer, improving its tack at low temperatures. When the content of the styrene-butadiene-styrene block copolymer (A2) is 70% by mass or less, the cohesive strength of the hot melt adhesive is improved, leading to improved holding power at room temperature, and its usability at low temperatures is also improved.

The styrene-isoprene-styrene block copolymer (A3) may be hydrogenated or not. It is, however, preferable that the styrene-isoprene-styrene block copolymer (A3) is not hydrogenated because the usability at low temperatures of the hot melt adhesive in which the styrene-isoprene-styrene block copolymer (A3) is not hydrogenated is excellent.

The styrene-isoprene-styrene block copolymer (A3) is commercially available. Examples of the commercially available styrene-isoprene-styrene block copolymer (A3) include “Quintac 3433N” available from Zeon Corporation and “D1161” available from Kraton Performance Polymers Inc.

The styrene content in the styrene-isoprene-styrene block copolymer (A3) preferably is 10-40% by mass, more preferably 11-35% by mass, and even more preferably 12-30% by mass. It is particularly preferable that the styrene content in the styrene-isoprene-styrene block copolymer (A3) is 13-25% by mass. When the styrene content in the styrene-isoprene-styrene block copolymer (A3) is 10% by mass or more, the holding power of the hot melt adhesive at room temperature is improved. When the styrene content in the styrene-isoprene-styrene block copolymer (A3) is 40% by mass or less, the tack of the hot melt adhesive at low temperatures is improved.

The viscosity of the styrene-isoprene-styrene block copolymer (A3) in a 25% toluene solution is preferably 100-3500 mPa s, and more preferably 400-2000 mPa s. When the viscosity of the styrene-isoprene-styrene block copolymer (A3) in a 25% toluene solution is 300 mPa s or higher, the holding power of the hot melt adhesive at room temperature is improved. When the viscosity of the styrene-isoprene-styrene block copolymer (A3) in a 25% toluene solution is 3500 mPa s or lower, the usability of the hot melt adhesive at low temperatures is improved.

The content of the styrene-isoprene-styrene block copolymer (A3) in the hot melt adhesive is preferably 30-70% by mass, and more preferably 40-60% by mass, relative to the total amount of the styrene-butadiene-styrene block copolymer (A1) and the styrenic block copolymer (A4). When the content of the styrene-isoprene-styrene block copolymer (A3) is 30% by mass or more, the hot melt adhesive becomes softer, improving its tack at low temperatures. When the content of the styrene-isoprene-styrene block copolymer (A3) is 70% by mass or less, the cohesive strength of the hot melt adhesive is improved, leading to improved holding power at room temperature, and its usability at low temperatures is also improved.

The content of the styrenic block copolymer (A4) in the hot melt adhesive is preferably 30-70% by mass, and more preferably 40-60% by mass, relative to the total amount of the styrene-butadiene-styrene block copolymer (A1) and the styrenic block copolymer (A4). When the content of the styrenic block copolymer (A4) is 30% by mass or more, the hot melt adhesive becomes softer, improving its tack at low temperatures. When the content of the styrenic block copolymer (A4) is 70% by mass or less, the cohesive strength of the hot melt adhesive is improved, leading to improved holding power at room temperature, and its usability at low temperatures is also improved.

The styrenic block copolymer (A) contains the styrene-butadiene-styrene block copolymer (A1) and, at least one styrenic block copolymer (A4) selected from the group consisting of the styrene-butadiene-styrene block copolymer (A2) and the styrene-isoprene-styrene block copolymer (A3). A styrenic block copolymer or copolymers other than the aforementioned styrenic block copolymers may also be contained. Such styrenic block copolymer is not specifically limited and examples include styrene-butadiene-styrene (SBS), styrene-isoprene-styrene (SIS), and their hydrogenated products. Examples of the hydrogenated products include styrene-butadiene/butylene-styrene (SBBS; a partially hydrogenated product of SBS), styrene-ethylene/butylene-styrene (SEBS; a partially hydrogenated product of SBS), styrene-ethylene/propylene-styrene (SEPS; a fully hydrogenated product of SIS), and styrene-ethylene-ethylene/propylene-styrene (SEEPS).

### Tackifier (B)

The hot melt adhesives of the present invention contain a tackifier (B). The tackifier (B) is not specifically limited and examples include a fully hydrogenated tackifier (B1), an end-block tackifier (B2), natural rosin, modified rosins, glycerol esters of natural rosin, glycerol esters of modified rosins, pentaerythritol esters of natural rosin, pentaerythritol esters of modified rosins, terpene resins, partially hydrogenated products obtained by adding hydrogen atoms to a part of the double bonds in phenolic modified terpene resins; petroleum resins such as C5 petroleum resins, C9 petroleum resins, C5C9 petroleum resins, and dicyclopentadiene-based petroleum resins; and partially hydrogenated petroleum resins obtained by adding hydrogen atoms to a part of the double bonds in any one of the aforementioned petroleum resins. The tackifier herein may be a single tackifier or a combination of two or more tackifiers.

The tackifier (B) is commercially available. Examples of the commercially available tackifiers other than the fully hydrogenated tackifier (B1) and the end-block tackifier (B2) include “Escorez 5600” available from Exxon Mobil Corp., “Marukaclear H” available from Maruzen Petrochemical Co., Ltd., “ARKON M-100” available from Arakawa Chemical Industries, Ltd., “I-MARV S-100” available from Idemitsu Kosan Co., Ltd., “ECR231C” available from Exxon Mobil Corp., “Regalite R7100” available from Eastman Chemical Company, “Regalite C6100” available from Eastman Chemical Company, and “SUKOREZ SU-400” available from Kolon Co., Ltd.

The tackifier other than the fully hydrogenated tackifier (B1) and the end-block tackifier (B2) is preferably one or more of non-hydrogenated tackifiers and partially hydrogenated tackifiers, and more preferably one or more of non-hydrogenated petroleum resins and partially hydrogenated petroleum resins because the usability at low temperatures and the thermal stability of the hot melt adhesive are excellent. Partially hydrogenated petroleum resins are particularly preferable.

The temperature for the ring-and-ball softening point of the tackifier other than the fully hydrogenated tackifier (B1) and the end-block tackifier (B2) is preferably 95° C. or higher, and more preferably 100° C. or higher because the hot melt adhesive has excellent adhesive strength and holding power at room temperature as well as excellent thermal stability. The temperature for the ring-and-ball softening point of the tackifier other than the fully hydrogenated tackifier (B1) and the end-block tackifier (B2) is preferably 125° C. or lower, and more preferably 120° C. or lower because the hot melt adhesive becomes softer, improving its usability at low temperatures. The temperature for the ring-and-ball softening point of the tackifier as used herein refers to a temperature determined according to JIS K2207.

The content of the tackifier (B) in the hot melt adhesive is 100-400 parts by mass, and preferably 200-350 parts by mass, relative to 100 parts by mass of the styrenic block copolymer (A). When the content of the tackifier (B) is 100 parts by mass or more, the adhesive strength of the hot melt adhesive at room temperature is improved. When the content of the tackifier (B) is 400 parts by mass or less, the hot melt adhesive becomes softer, improving its tack at low temperatures.

The content of the tackifier other than the fully hydrogenated tackifier (B1) and the end-block tackifier (B2) in the hot melt adhesive is preferably 200-400 parts by mass, and more preferably 250-350 parts by mass, relative to 100 parts by mass of the styrenic block copolymer (A). When the content of the tackifier other than the fully hydrogenated tackifier (B1) and the end-block tackifier (B2) is 200 parts by mass or more, the holding power of the hot melt adhesive at room temperature is improved. When the content of the tackifier other than the fully hydrogenated tackifier (B1) and the end-block tackifier (B2) is 400 parts by mass or less, the tack of the hot melt adhesive at low temperatures is improved.

It is preferable that the tackifier (B) contains the fully hydrogenated tackifier (B1). It is more preferable that the tackifier (B) contains the fully hydrogenated tackifier (B1), and one or more tackifiers other than the fully hydrogenated tackifier (B1) and the end-block tackifier (B2). When the hot melt adhesive contains the fully hydrogenated tackifier (B1), and the tackifier other than the fully hydrogenated tackifier (B1) and the end-block tackifier (B2), the adhesive strength and holding power at room temperature as well as the tack at low temperatures are improved. The “fully hydrogenated tackifier (B1)” refers to a tackifier in which hydrogen atoms have been added to substantially all the double bonds in the tackifier. The fully hydrogenated tackifier (B1) is not specifically limited and examples include fully hydrogenated petroleum resins obtained by adding hydrogen atoms to all double bonds in petroleum resins such as C5 petroleum resins, C9 petroleum resins, C5C9 petroleum resins, and dicyclopentadiene-based petroleum resins, and fully hydrogenated terpene resins obtained by adding hydrogen atoms to all double bonds in terpene resins. Fully hydrogenated petroleum resins are preferable.

The fully hydrogenated tackifier (B1) is commercially available. Examples of the commercially available fully hydrogenated tackifier (B1) include “ARKON P-90” available from Arakawa Chemical Industries, Ltd., “ARKON P-100” available from Arakawa Chemical Industries, Ltd., “I-MARV P-100” available from Idemitsu Kosan Co., Ltd., and “CLEARON P85” available from YASUHARA CHEMICAL CO., LTD.

The ring-and-ball softening point of the fully hydrogenated tackifier (B1) is preferably 100° C. or lower, and more preferably 95° C. or lower. When the ring-and-ball softening point of the fully hydrogenated tackifier (B1) is 100° C. or lower, the tack of the hot melt adhesive at low temperatures is improved. The ring-and-ball softening point of the fully hydrogenated tackifier (B1) is preferably 80° C. or higher, and more preferably 85° C. or higher. When the ring-and-ball softening point of the fully hydrogenated tackifier (B1) is 80° C. or higher, the adhesive strength of the hot melt adhesive at room temperature is improved.

The content of the fully hydrogenated tackifier (B1) in the hot melt adhesive is preferably 10-100 parts by mass, and more preferably 30-70 parts by mass, relative to 100 parts by mass of the styrenic block copolymer (A). When the content of the fully hydrogenated tackifier (B1) is 10 parts by mass or more, the cohesive strength of the hot melt adhesive is improved, leading to improved holding power at room temperature, and its usability at low temperatures is also improved. When the content of the fully hydrogenated tackifier (B1) is 100 parts by mass or less, the hot melt adhesive becomes softer, improving its tack at low temperatures.

It is preferable that the tackifier (B) contains the end-block tackifier (B2). It is more preferable that the tackifier (B) contains the end-block tackifier (B2) and, the tackifier other than the fully hydrogenated tackifier (B1) and the end-block tackifier (B2). When the hot melt adhesive contains the end-block tackifier (B2), and the tackifier other than the fully hydrogenated tackifier (B1) and the end-block tackifier (B2), the adhesive strength and holding power at room temperature as well as the tack at low temperatures are improved.

The end-block tackifier (B2) is a homopolymer of a styrenic monomer, a copolymer of different styrenic monomers, or a copolymer of a styrenic monomer and another monomer that can be copolymerized with the styrenic monomer. The end-block tackifier (B2) is miscible with styrene blocks of the styrenic block copolymer.

The styrenic monomer is not specifically limited and examples include styrene, alpha-methyl styrene, indene, vinyl toluene, and derivatives thereof. The styrenic monomer may be a single styrenic monomer or a combination of two or more styrenic monomers.

The ring-and-ball softening point of the end-block tackifier (B2) is preferably 95° C. or lower, and more preferably 90° C. or lower. When the ring-and-ball softening point is 95° C. or lower, the melt viscosity of the hot melt adhesive is reduced, improving its usability at low temperatures without losing its holding power at room temperature. The ring-and-ball softening point of the end-block tackifier (B2) is preferably 80° C. or higher, and more preferably 85° C. or higher. When the ring-and-ball softening point of the end-block tackifier (B2) is 80° C. or higher, the holding power of the hot melt adhesive at room temperature is improved.

The end-block tackifier (B2) is commercially available. Examples of the commercially available end-block tackifier (B2) include “Kristalex 3085” available from Eastman Chemical Company and “FTR6100” available from Mitsui Chemicals, Inc.

The content of the end-block tackifier (B2) in the hot melt adhesive is preferably 5-35 parts by mass, and more preferably 10-25 parts by mass, relative to 100 parts by mass of the styrenic block copolymer (A). When the content of the end-block tackifier (B2) is 5 parts by mass or more, the holding power of the hot melt adhesive at room temperature is improved. When the content of the end-block tackifier (B2) is 35 parts by mass or less, the hot melt adhesive becomes softer, improving its tack at low temperatures.

When the tackifier (B) contains two or more different tackifiers such as the fully hydrogenated tackifier (B1) and/ or the end-block tackifier (B2), it is necessary to adjust the amount of the tackifiers so that the total amount of the all tackifiers should be in the range of 100-400 parts by mass relative to 100 parts by mass of the styrenic block copolymer (A).

### Plasticizer

The hot melt adhesive contains a plasticizer (C). The plasticizer (C) is not specifically limited and examples include process oils such as paraffinic process oils, naphthenic process oils, and aromatic process oils. The paraffinic process oils and the naphthenic process oils are particularly preferable, but the naphthenic process oils are more preferable because the thermal stability, and the adhesive strength and holding power at room temperature of the hot melt adhesive are improved. The plasticizer may be a single plasticizer or a combination of two or more plasticizers.

The paraffinic process oils contain one or more chain aliphatic hydrocarbons. The number of carbon atoms in the chain aliphatic hydrocarbon is not specifically limited. The number is, however, preferably 16-40, and more preferably 20-30.

The number average molecular weight (Mn) of the paraffinic process oil is preferably 100-1500, more preferably 250-1000. When the number average molecular weight (Mn) is 100 or more, the cohesive strength of the hot melt adhesive is improved, leading to improved holding power at room temperature, and its usability at low temperatures is also improved. When the number average molecular weight (Mn) is 1500 or less, the usability of the hot melt adhesive at low temperatures is improved.

The paraffinic process oil is commercially available. Examples of the commercially available paraffinic process oils include “NA Solvent” available from NOF Corporation, “PW-380,” “Diana Fresia S32,” “PS-32,” “Diana Process Oil PS-32,” and “IP-Solvent 2835” available from Idemitsu Kosan Co., Ltd., and “Neochiozol” available from Sanko Chemical Industry Co., Ltd.

The naphthenic process oil is not specifically limited as long as it contains an alicyclic hydrocarbon. However, the number of carbon atoms in the alicyclic hydrocarbon of the naphthenic process oil is preferably 3 or more, and more preferably 3-8.

The number average molecular weight (Mn) of the naphthenic process oil is preferably 100-1500, more preferably 250-1000. When the number average molecular weight (Mn) is 100 or more, the cohesive strength of the hot melt adhesive is improved, leading to improved holding power at room temperature, and its usability at low temperatures is also improved. When the number average molecular weight (Mn) is 1500 or less, the usability of the hot melt adhesive at low temperatures is improved.

The naphthenic process oil is commercially available. Examples of the commercially available naphthenic process oils include “Diana Fresia N28,” “Diana Fresia U46,” and “Diana Process Oil NR” available from Idemitsu Kosan Co., Ltd., and “Shellflex 371N” available from Shell Chemicals.

The number average molecular weight (Mn) of the paraffinic and naphthenic process oils as used herein refers to a value obtained by standardizing a value measured by gel permeation chromatography equipment with polystyrene standard.

The content of the plasticizer (C) in the hot melt adhesive is preferably 25-250 parts by mass, more preferably 50-150 parts by mass, relative to 100 parts by mass of the styrenic block copolymer (A). When the content of the plasticizer is 25 parts by mass or more, the melt viscosity of the hot melt adhesive is reduced, improving the tack and usability at low temperatures. When the content of the plasticizer is 250 parts by mass or less, the holding power of the hot melt adhesive at room temperature is improved.

The hot melt adhesives of the present invention can contain a wax as long as their properties are not impaired. The wax is not specifically limited and examples include animal waxes such as shellac wax and beeswax, plant waxes such as carnauba wax and Japan wax, mineral waxes such as paraffin wax and macro-crystalline wax, and synthetic waxes such as Fischer-Tropsch waxes and polyolefin waxes including ethylene-vinyl acetate (EVA) copolymer waxes. The wax is preferably a polyolefin wax and, more preferably an ethylene-vinyl acetate (EVA) copolymer wax because they do not lower the thermal stability of the hot melt adhesives and the adhesive strength of the hot melt adhesive at room temperature is excellent.

The wax is commercially available. Examples of the commercially available polyolefin waxes include “A-C7” available from Honeywell International, Inc., and “VISCOWAX 122” available from Innospec Leuna GmbH. Examples of the commercially available ethylene-vinyl acetate (EVA) copolymer waxes include “A-C400” and “A-C405S” available from Honeywell International, Inc., and “VISCOWAX 334” and “VISCOWAX 343” available from Honeywell International, Inc.

It is preferable that the hot melt adhesives contain an antioxidant as long as their properties are not impaired. Examples of the antioxidants include hindered phenol type antioxidants such as 2,6-di-t-butyl-4-methylphenol, n-octadecyl-3-(4′-hydroxy-3′,5′-di-t-butyl-phenyl) propionate, 2,2′-methylenebis(4-methyl-6-t-butylphenol), 2,2′-methylenebis(4-ethyl-6-t-butylphenol), 2,4-bis(octylthiomethyl)-o-cresol, 2-t-butyl-6-(3-t-butyl-2-hydroxy-5-methylbenzyl)-4-methylphenyl acrylate, 2,4-di-t-amyl-6-[1(3,5-di-t-amyl-2-hydroxyphenyl)ethyl]phenyl acrylate, 2-[1-(2-hydroxy-3,5-di-tert-pentylphenyl)]acrylate, tetrakis[methylene-3-(3,5-di-t-butyl-4-hydroxyphenyl)propionate]methane; sulfur antioxidants such as dilauryl thiodipropionate, lauryl stearyl thiodipropionate, and pentaerythritol tetrakis(3-laurylthiopropionate); and phosphorus antioxidants such as tris(nonylphenyl)phosphite, and tris(2,4-di-t-butyl-phenyl) phosphite. The antioxidant herein may be a single antioxidant or a combination of two or more antioxidants.

The hot melt adhesives may contain a UV absorber as long as their properties are not impaired. Examples of the UV absorbers include benzotriazole UV absorbers such as 2-(2′-hydroxy-5′-methylphenyl)benzotriazole, 2-(2′-hydroxy-3′,5′-t-butylphenyl)benzotriazole, and 2-(2′-hydroxy-3′,5′-di-t-butyl-phenyl)-5-chlorobenzotriazole; benzophenone UV absorbers such as 2-hydroxy-4-methoxy benzophenone; salicylic acid ester UV absorbers, cyanoacrylate UV absorbers; and hindered amine light stabilizers. The UV absorber herein may be a single UV absorber or a combination of two or more UV absorbers.

The hot melt adhesives may contain a liquid rubber as long as their properties are not impaired. Examples of the liquid rubbers include liquid polybutene, liquid polybutadiene, liquid polyisoprene, and their hydrogenated resins. The liquid rubber herein may be a single liquid rubber or a combination of two or more liquid rubbers.

The melt viscosity of the hot melt adhesive at 120° C. is 6000 mPa s or lower, preferably 5500 mPa s or lower, and more preferably 5000 mPa s or lower. When the melt viscosity of the hot melt adhesive at 120° C. is 6000 mPa s or lower, the hot melt adhesive has excellent usability at low temperatures. The melt viscosity of the hot melt adhesive at 120° C. is preferably 1500 mPa s or higher, and more preferably 2000 mPa s or higher. When the melt viscosity of the hot melt adhesive at 120° C. is 1500 mPa s or higher, the holding power of the hot melt adhesive at room temperature is improved. The melt viscosity of the hot melt adhesive at 120° C. as used herein is values determined by melting the hot melt adhesive with heat and measuring a viscosity of the molten hot melt adhesive at 120° C. using a Brookfield RVT viscometer (spindle #27).

The hot melt adhesive of the present invention can be used to bond two materials in the following exemplified manner. First, the hot melt adhesive is heated into a molten state. Next, the molten hot melt adhesive is applied to one of the materials. The other material is put over the adhesive-applied material. Then, the hot melt adhesive is cooled and solidified, thereby bonding the two materials together.

A method of applying the molten hot melt adhesive to a material is not specifically limited and known methods are used. Examples include slot coating, roll coating, swirl pattern application, omega-shaped pattern application, controlled seaming, curtain spraying, and dot pattern application. Swirl pattern application, omega-shaped pattern application, and curtain spraying are preferable because the disposable products obtained using these methods have good textures.

The hot melt adhesives of the present invention can be used suitably for the production of disposable products. The disposable products are not specifically limited and examples include so-called sanitary materials such as disposable diapers, sanitary napkins, absorbent pads for animals, hospital gowns, and surgical white coats.

The disposable products are not specifically limited and have a first component, a second component, and a hot melt adhesive of the present invention. The first component is bonded and integrated with the second component by the hot melt adhesive.

The first and second components may be any components that are used for the disposable products, and examples include polyolefin resin films, non-woven fabrics, woven fabrics, natural rubbers, and hydrophilic porous substrates. They may be used singly or in combination of each other. Examples of the hydrophilic porous substrates include porous substrates containing cellulose or cotton, and hydrophilic-treated porous substrates. Examples of the porous substrates containing cellulose or cotton include tissue papers. Examples of the hydrophilic-treated porous substrates include hydrophilic-treated non-woven and woven fabrics.

### EXAMPLES

The present invention is described more specifically by Examples, but the present invention is not limited thereto.

In the Examples and Comparative examples described below, the styrenic block copolymer (A), the tackifier (B), the plasticizer (C), the wax, and the antioxidant used for the production of hot melt adhesives are described in detail below.

### Styrenic Block Copolymer (A)

### Styrenic Block Copolymer (A1)

Styrene-butadiene-styrene (SBS) block copolymer (A1) (“Asaprene T-439” manufactured by Asahi Kasei Chemicals Corporation; non-hydrogenated; styrene content: 45% by mass; viscosity in a 25% toluene solution: 170 mPa s)

### Styrenic Block Copolymer (A2)

Styrene-butadiene-styrene (SBS) block copolymer (A2-1) (“Asaprene T-438” manufactured by Asahi Kasei Chemicals Corporation; non-hydrogenated; styrene content: 35% by mass; viscosity in a 25% toluene solution: 470 mPa s)

Styrene-butadiene-styrene (SBS) block copolymer (A2-2) (“Asaprene T-432” manufactured by Asahi Kasei Chemicals Corporation; non-hydrogenated; styrene content: 30% by mass; viscosity in a 25% toluene solution: 3100 mPa s)

### Styrene-Isoprene-Styrene Block Copolymer (A3)

Styrene-isoprene-styrene (SIS) block copolymer (A3) (“Quintac 3433N” manufactured by Zeon Corporation; non-hydrogenated; styrene content: 15% by mass; viscosity in a 25% toluene solution: 810 mPa s)

### Tackifier

Partially hydrogenated petroleum resin (B-1) (“ARKON M-100” manufactured by Arakawa Chemical Industries, Ltd.; an aliphatic petroleum hydrocarbon resin; ring-and-ball softening point: 100° C.)

Partially hydrogenated petroleum resin (B-2) (“I-MARV S-110” manufactured by Idemitsu Kosan Co., Ltd.; an aliphatic-aromatic petroleum hydrocarbon resin; ring-and-ball softening point: 110° C.)

### Fully Hydrogenated Tackifier (B1)

Fully hydrogenated petroleum resin (B1-1) (“ARKON P-90” manufactured by Arakawa Chemical Industries, Ltd.; an aliphatic petroleum hydrocarbon resin; ring-and-ball softening point: 90° C.)

Fully hydrogenated petroleum resin (B1-2) (“I-MARV P-100” manufactured by Idemitsu Kosan Co., Ltd.; an aliphatic-aromatic petroleum hydrocarbon resin; ring-and-ball softening point: 100° C.)

### End-Block Tackifier (B2)

End-block tackifier (B2-1) (“Kristalex 3085” manufactured by Eastman Chemical Company; a styrene-alpha-methyl styrene copolymer; non-hydrogenated; ring-and-ball softening point: 85° C.)

End-block tackifier (B2-2) (“FTR6100” manufactured by Mitsui Chemicals, Inc.; a copolymer of a styrene monomer and an aliphatic monomer; ring-and-ball softening point: 95° C.)

### Plasticizer

Paraffinic process oils (C1) (“Diana Process Oil PS-32” manufactured by Idemitsu Kosan Co., Ltd.; number average molecular weight: 980)

Naphthenic process oils (C2) (“Shellflex 371N” manufactured by Shell Chemicals; number average molecular weight: 1500)

### Wax

Ethylene-vinyl acetate (EVA) copolymer wax (a graft copolymer with a polyethylene backbone and branched side chains of vinyl acetate; melting point: 94° C.; “A-C405S” manufactured by Honeywell International, Inc.)

### Antioxidant

Hindered phenol antioxidant (“IRGANOX 1010” manufactured by BASF)

### Examples 1-12 and Comparative Examples 1-3

The styrene-butadiene-styrene (SBS) block copolymers (A1), (A2-1), and (A2-2), the styrene-isoprene-styrene (SIS) block copolymer (A3), the partially hydrogenated petroleum resins (B-1) and (B-2), the fully hydrogenated petroleum resins (B1-1) and (B1-2), the end-block tackifiers (B2-1) and (B2-2), the paraffinic process oil (C1), the naphthenic process oil (C2), the ethylene-vinyl acetate (EVA) copolymer wax and the hindered phenol antioxidant as described above were placed into a mixing kneader equipped with a heater in the amounts indicated in Tables 1 and 2, and kneaded with heating at 145° C. for 90 min. to produce hot melt adhesives.

### Evaluations

Melt viscosities of the hot melt adhesives at 120° C. were determined in the manner as described above. Viscosity retention indexes, usability at low temperatures, adhesive strength and holding power at room temperature, and tack at low temperatures of the hot melt adhesives were determined as described below. The results are given in Tables 1 and 2.

### Viscosity Retention Index

Melt viscosities (i.e., melt viscosities before aging) of the hot melt adhesives at 120° C. were determined. The hot melt adhesives were then placed in glass bottles and allowed to stand at 180° C. for 3 days. Thereafter, the hot melt adhesives were heated to 120° C. and the melt viscosities (i.e., melt viscosities after aging) of the molten hot melt adhesives at 120° C. were determined using a Brookfield RVT viscometer (spindle #27) and viscosity retention indexes were calculated according to the following equation.

Viscosity retention index (%) =100 ×(melt viscosity after aging)/(melt viscosity before aging)

### Usability at Low Temperatures

The hot melt adhesives were melted by heating them to 120° C. and applied by omega-shaped pattern application to polyethylene terephthalate films in a coating amount of 6 g/m² each at a line speed of 300 m/min. Subsequently, a polyethylene terephthalate film was placed over each of the adhesive-coated surfaces of the polyethylene terephthalate films to produce laminates. Each of the laminates was compressed in the thickness direction at 23° C. and with a pressure of 50 gf/cm² (4903 Pa) for 0.01 seconds to bond the films. The hot melt adhesive was visually observed through the polyethylene terephthalate film. When the omega pattern of the applied adhesive was clearly visible, the usability at low temperatures was determined as “EXCELLENT.” When the omega pattern of the applied adhesive was visible with some irregular spread, the usability at low temperatures was determined as “GOOD.” When the omega pattern of the applied adhesive became irregular, the usability at low temperatures were determined as “POOR.”

### Adhesive Strength at Room Temperature

The hot melt adhesives were melted by heating them to 120° C. and applied by slot coating to polyethylene terephthalate films in a coating amount of 50 g/m² over a width of 25 mm. Subsequently, a polyethylene terephthalate film having a release-treated surface was placed over each of the adhesive-coated surfaces of the polyethylene terephthalate films to produce laminates. Each of the laminates was compressed in the thickness direction at 23° C. and with a pressure of 50 gf/cm² (4903 Pa) for 0.01 seconds to bond the films. The release-treated polyethylene terephthalate films were separated from the adhesive-coated polyethylene terephthalate films and an air-impermeable polyethylene film was placed over each of the adhesive-coated surfaces of the polyethylene terephthalate films. A 2-kg roller was passed back and forth one time over each of the polyethylene films to produce test specimens.

The test specimens were allowed to stand at 23° C. in an atmosphere of 50% relative humidity for 24 hours to cool and solidify the hot melt adhesives. The test specimens were subjected to a T-peel test at 23° C. by pulling apart the films in the vertical direction to the surfaces of the laminates at a rate of 300 mm/min. and a peel adhesion (×10⁻²N/25 mm) was measured.

### Holding Power at Room Temperature

The hot melt adhesives were melted by heating them to 120° C. and applied by slot coating to polyethylene terephthalate films in a coating amount of 50 g/m² over a width of 25 mm. Subsequently, a polyethylene terephthalate film having a release-treated surface was placed over each of the adhesive-coated surfaces of the polyethylene terephthalate films to produce laminates. Each of the laminates was compressed in the thickness direction at 23° C. and with a pressure of 50 gf/cm² (4903 Pa) for 0.01 seconds to bond the films. The release-treated polyethylene terephthalate film was separated from the adhesive-coated polyethylene terephthalate films and an air-impermeable polyethylene film was placed over each of the adhesive-coated surfaces of the polyethylene terephthalate films. The contact area between the hot melt adhesive and the polyethylene film had a flat rectangular shape of 10 mm in length and 25 mm in width. Subsequently, a 2-kg roller was passed back and forth one time over each of the polyethylene films to produce test specimens. The test specimens were placed so that the detached portions of their polyethylene films are in parallel with the gravity direction. A 1-kg weight was attached to each of the lower edges of the air-impermeable polyethylene films of the test specimens and the test specimens were then left in an atmosphere of 40° C. The elapsed time for the air-impermeable polyethylene film to completely separate from the hot melt adhesive and fall down with the weight after the weight was attached to the lower edge of the polyethylene film of the test specimen was recorded as a “ holding power at room temperature.”

### Tack at Low Temperatures

A loop tack test was performed for the hot melt adhesives at 10° C. according to the following manner to evaluate tack at low temperatures.

The hot melt adhesives were melted by heating them to 120° C. and applied by slot coating to polyethylene terephthalate films in a coating amount of 50 g/m² over a width of 25 mm. Subsequently, a polyethylene terephthalate film having a release-treated surface was placed over each of the adhesive-coated surfaces of the polyethylene terephthalate films to produce laminates. Each of the laminates was compressed in the thickness direction at 23° C. and with a pressure of 50 gf/cm² (4903 Pa) for 0.01 seconds to bond the films. The laminated sheet was cut into rectangular strips (25 mm in width by 230 mm in length). The polyethylene terephthalate film having the release-treated surface was removed from each strip and each strip was formed into a loop with the hot melt adhesive outside. Both ends of the strip were overlapped with each other and the overlapped portion was held using a chuck to form a loop-shaped test specimen (80 mm in major diameter and 180 mm in perimeter length).

Next, each test specimen was hung at 10° C. directing the overlapped portion of the test specimen held by the chuck above and the curved portion of the test specimen below. A polyethylene plate was placed with its upper surface horizontal under the test specimens. The test specimens were lowered at a rate of 300 m/min. until the distance between the chuck and the upper surface of the polyethylene plate reaches 50 mm. Immediately thereafter, the test specimens were pulled up at a rate of 300 m/min. and separated from the polyethylene plate. A peel strength [N/25 mm] at that time was measured.

TABLE 1

| | | | Example 1 | Example 2 | Example 3 | Example 4 |
|---|---|---|---|---|---|---|
| formulation | styrenic | styrene-butadiene-styrene block copolymer (A1) | 50 | 70 | 30 | 50 |
| (parts by | block | styrene-butadiene-styrene block copolymer (A2-1) | 50 | 30 | 70 | 50 |
| mass) | copolymer | styrene-butadiene-styrene block copolymer (A2-2) | 0 | 0 | 0 | 0 |
| | (A) | styrene-isoprene-styrene block copolymer (A3) | 0 | 0 | 0 | 0 |
| | tackifier (B) | partially hydrogenated petroleum resin (B-1) | 270 | 270 | 270 | 270 |
| | | partially hydrogenated petroleum resin (B-2) | 0 | 0 | 0 | 0 |
| | | fully hydrogenated petroleum resin (B1-1) | 50 | 50 | 50 | 0 |
| | | fully hydrogenated petroleum resin (B1-2) | 0 | 0 | 0 | 50 |
| | | end-block tackifier (B2-1) | 0 | 0 | 0 | 0 |
| | | end-block tackifier (B2-2) | 0 | 0 | 0 | 0 |
| | plasticizer | paraffinic process oil (C1) | 100 | 100 | 100 | 100 |
| | (C) | naphthenic process oil (C2) | 0 | 0 | 0 | 0 |
| | wax | ethylene-vinyl acetate (EVA) copolymer wax | 0 | 0 | 0 | 0 |
| | Antioxidant | hindered phenol antioxidant | 3 | 3 | 3 | 3 |
| evaluation | melt viscosity at 120° C. (mPa s) | | 3600 | 3000 | 3900 | 3700 |
| | viscosity retention index (%) | | 82.6 | 82.9 | 82.7 | 82.6 |
| | usability at low temperatures | | Excellent | Excellent | Excellent | Excellent |
| | adhesive strength at room temperature (N/25 mm) | | 12.4 | 12.0 | 18.2 | 12.3 |
| | holding power at room temperature (min.) | | 45 | 48 | 40 | 46 |
| | tack at low temperatures (N/25 mm) | | 1.1 | 1.0 | 1.2 | 1.0 |
| | | | Example 5 | Example 6 | Example 7 | Example 8 |
| formulation | styrenic | styrene-butadiene-styrene block copolymer (A1) | 50 | 50 | 50 | 50 |
| (parts by | block | styrene-butadiene-styrene block copolymer (A2-1) | 50 | 50 | 50 | 50 |
| mass) | copolymer | styrene-butadiene-styrene block copolymer (A2-2) | 0 | 0 | 0 | 0 |
| | (A) | styrene-isoprene-styrene block copolymer (A3) | 0 | 0 | 0 | 0 |
| | tackifier (B) | partially hydrogenated petroleum resin (B-1) | 320 | 320 | 300 | 300 |
| | | partially hydrogenated petroleum resin (B-2) | 0 | 0 | 0 | 0 |
| | | fully hydrogenated petroleum resin (B1-1) | 0 | 0 | 0 | 0 |
| | | fully hydrogenated petroleum resin (B1-2) | 0 | 0 | 0 | 0 |
| | | end-block tackifier (B2-1) | 0 | 0 | 20 | 0 |
| | | end-block tackifier (B2-2) | 0 | 0 | 0 | 20 |
| | plasticizer | paraffinic process oil (C1) | 100 | 0 | 100 | 100 |
| | (C) | naphthenic process oil (C2) | 0 | 100 | 0 | 0 |
| | wax | ethylene-vinyl acetate (EVA) copolymer wax | 0 | 0 | 0 | 0 |
| | Antioxidant | hindered phenol antioxidant | 3 | 3 | 3 | 3 |

| | | | | | |
|---|---|---|---|---|---|
| evaluation | melt viscosity at 120° C. (mPa s) | 4000 | 4200 | 3800 | 3700 |
| | viscosity retention index (%) | 81.0 | 81.4 | 79.5 | 80.0 |
| | usability at low temperatures | Excellent | Excellent | Excellent | Excellent |
| | adhesive strength at room temperature (N/25 mm) | 10.3 | 11.1 | 13.4 | 13.3 |
| | holding power at room temperature (min.) | 35 | 38 | 45 | 46 |
| | tack at low temperatures (N/25 mm) | 0.8 | 0.8 | 1.2 | 1.1 |

TABLE 2

| | | | | Example | Example | Example |
|---|---|---|---|---|---|---|
| | | | Example 9 | 10 | 11 | 12 |
| formulation | styrenic block | styrene-butadiene-styrene block copolymer (A1) | 50 | 70 | 50 | 50 |
| (parts by | copolymer (A) | styrene-butadiene-styrene block copolymer (A2-1) | 50 | 0 | 50 | 0 |
| mass) | | styrene-butadiene-styrene block copolymer (A2-2) | 0 | 30 | 0 | 0 |
| | | styrene-isoprene-styrene block copolymer (A3) | 0 | 0 | 0 | 50 |
| | tackifier (B) | partially hydrogenated petroleum resin (B-1) | 0 | 270 | 320 | 270 |
| | | partially hydrogenated petroleum resin (B-2) | 270 | 0 | 0 | 0 |
| | | fully hydrogenated petroleum resin (B1-1) | 50 | 50 | 0 | 50 |
| | | fully hydrogenated petroleum resin (B1-2) | 0 | 0 | 0 | 0 |
| | | end-block tackifier (B2-1) | 0 | 0 | 0 | 0 |
| | | end-block tackifier (B2-2) | 0 | 0 | 0 | 0 |
| | plasticizer (C) | paraffinic process oil (C1) | 100 | 100 | 100 | 100 |
| | | naphthenic process oil (C2) | 0 | 0 | 0 | 0 |
| | wax | ethylene-vinyl acetate (EVA) copolymer wax | 0 | 0 | 10 | 0 |
| | antioxidant | hindered phenol antioxidant | 3 | 3 | 3 | 3 |

| | | | | | |
|---|---|---|---|---|---|
| evaluation | melt viscosity at 120° C. (mPa s) | 3500 | 5300 | 3800 | 4100 |
| | viscosity retention index (%) | 83.1 | 81.1 | 82.5 | 72.0 |
| | usability at low temperatures | Excellent | Good | Excellent | Excellent |
| | adhesive strength at room temperature (N/25 mm) | 14.8 | 15.0 | 10.2 | 17.4 |
| | holding power at room temperature (min.) | 52 | 45 | 39 | 34 |
| | tack at low temperatures (N/25 mm) | 0.9 | 0.9 | 0.9 | 1.3 |

| | | | Comparative | Comparative | Comparative |
|---|---|---|---|---|---|
| | | | example 1 | example 2 | example 3 |
| formulation | styrenic | styrene-butadiene-styrene block copolymer (A1) | 0 | 100 | 80 |
| (parts by mass) | block copolymer | styrene-butadiene-styrene block copolymer (A2-1) | 70 | 0 | 20 |
| | (A) | styrene-butadiene-styrene block copolymer (A2-2) | 30 | 0 | 0 |
| | | styrene-isoprene-styrene block copolymer (A3) | 0 | 0 | 0 |
| | tackifier (B) | partially hydrogenated petroleum resin (B-1) | 270 | 270 | 320 |
| | | partially hydrogenated petroleum resin (B-2) | 0 | 0 | 0 |
| | | fully hydrogenated petroleum resin (B1-1) | 50 | 50 | 0 |
| | | fully hydrogenated petroleum resin (B1-2) | 0 | 0 | 0 |
| | | end-block tackifier (B2-1) | 0 | 0 | 0 |
| | | end-block tackifier (B2-2) | 0 | 0 | 0 |
| | plasticizer (C) | paraffinic process oil (C1) | 100 | 100 | 100 |
| | | naphthenic process oil (C2) | 0 | 0 | 0 |
| | wax | ethylene-vinyl acetate (EVA) copolymer wax | 0 | 0 | 0 |
| | antioxidant | hindered phenol antioxidant | 3 | 3 | 3 |

| | | | | |
|---|---|---|---|---|
| evaluation | melt viscosity at 120° C. (mPa s) | 6500 | 2000 | 2800 |
| | viscosity retention index (%) | 83.4 | 83.1 | 83.0 |
| | usability at low temperatures | Poor | Good | Good |
| | adhesive strength at room temperature (N/25 mm) | 12.2 | 16.5 | 15.2 |
| | holding power at room temperature (min.) | 40 | 38 | 40 |
| | tack at low temperatures (N/25 mm) | 0.3 | 0.1 | 0.2 |

### INDUSTRIAL APPLICABILITY

The present invention is low-temperature hot melt adhesives that can be applied at relatively low temperatures, with good tack at low temperatures and with adhesive strength and holding power at room temperature not being impaired. The hot melt adhesives of the present invention can be used suitably for the production of disposable products such as disposable diapers.

1. A hot melt adhesive comprising:
100 parts by mass of a styrenic block copolymer (A), the styrenic block copolymer (A) comprising
a styrene-butadiene-styrene block copolymer (A1) having styrene content of 40-50% by mass and a viscosity in a 25% toluene solution of 100-300 mPa s, and
at least one styrenic block copolymer (A4) selected from the group consisting of a styrene-butadiene-styrene block copolymer (A2) having a styrene content of 15-40% by mass and a viscosity in a 25% toluene solution of 300-3500 mPa s, and a styrene-isoprene-styrene block copolymer (A3), wherein
(A1) is 30-70% by mass of the total amount of the styrene block copolymers (A1)plus(A4) ;
100-400 parts by mass of a tackifier (B); and
a plasticizer (C); wherein
the hot melt adhesive has a melt viscosity of 6000 mPa s or lower at 120° C.

2. The hot melt adhesive according to claim 1, wherein the tackifier (B) comprises 10-100 parts by mass of a fully hydrogenated tackifier (B1) having a ring-and-ball softening point of 100° C. or lower, relative to 100 parts by mass of the styrenic block copolymer (A).

3. The hot melt adhesive according to claim 1, wherein the tackifier (B) comprises an end-block tackifier (B2) having a ring-and-ball softening point of 95° C. or lower.

4. A disposable product made by using a hot melt adhesive according to claim 1.

5. The hot melt adhesive according to claim 2, wherein the tackifier (B) comprises an end-block tackifier (B2) having a ring-and-ball softening point of 95° C. or lower.

6. A disposable product made by using a hot melt adhesive according to claim 2.

7. A disposable product made by using a hot melt adhesive according to claim 3.

8. A disposable product made by using a hot melt adhesive according to claim 5.
